(19) United States
(12) Patent Application Publication
(10) Pub. No.: US 2004/0067204 A1
(43) Pub. Date: Apr. 8, 2004

(54) Use of antioxidants in means of treating halitosis

(76) Inventor: Florian Wolf, Hamburg (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/450,388

(22) PCT Filed: Dec. 3, 2001
(86) PCT No.: PCT/EP01/14099

(30) Foreign Application Priority Data
Dec. 15, 2000 (DE) 100 62 770.6

(51) Int. Cl. A61K 7/16

(52) U.S. Cl. 424/49

(57) ABSTRACT

Antioxidants or antioxidant-contaming preparations for producing compositions are used for eliminating halitosis or preventing the formation of halitosis. According to the invention, the antioxidants used can be all antioxidants which are suitable or customary for oral hygiene applications. In particular, vitamins and coenzyme come into consideration as antioxidants. Those which also come into consideration are, for example, amino acids, imidazoles, peptides, fatty acids, metal compounds and derivatives of these compounds (for example salts, esters, ethers, sugars, nucleotides, nucleosides).

The use of antioxidants in the oral and throat cavity is known in principle from the prior art. Thus JP 61286314 discloses the use of vitamins, for example vitamin E, vitamin B6, for treating paradontosis, in particular gingivitis.

In addition, WO 94/06418 discloses the use of coenzyme Q 10 for the therapeutic treatment of paradontosis.

To control bad breath (halitosis), according to the prior art, bactericidal compositions (for example Triclosan) or aroma compounds (menthol, menthol derivatives or mint aromas) are used. These compositions have the disadvantage that they are only effective against bad breath caused by bacteria.

It is an object of the present invention, therefore, to provide a composition which no longer has the disadvantages described in the prior art.

This object is achieved according to the invention by using antioxidants or antioxidant-containing preparations for producing compositions for eliminating halitosis or preventing the formation of halitosis.

According to the invention, the antioxidants used can be all antioxidants which are suitable or customary for oral hygiene applications. In particular, vitamins and coenzyme come into consideration as antioxidants. Those which also come into consideration are, for example, amino acids, imidazoles, peptides, fatty acids, metal compounds and derivatives of these compounds (for example salts, esters, ethers, sugars, nucleotides, nucleosides).

Preferably, the antioxidants are selected from the group consisting of amino acids (for example glycine, histidine, tyrosine, tryptophan) and derivatives thereof, imidazoles (for example urocanic acid) and derivatives thereof, peptides such as D,L-carnosine, D-carnosine, L-carnosine and derivatives thereof (for example anserine), carotenoids, carotenes (for example α-carotene, β-carotene, lycopene) and derivatives thereof, lipoic acid and derivatives thereof (for example dihydrolipoic acid), aurothioglucose, propylthiouracil and other thiols (for example thioredoxin, glutathione, cysteine, cystine, cystamine and glycosyl, N-acetyl, ethyl, propyl, amyl, butyl and lauryl, palmitoyl, oleyl, α-linoleyl, γ-linoleyl, cholesteryl, glyceryl, and oligoglyceryl esters thereof) and salts thereof, dilauryl thiodipropionate, disteaiyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoxirnine, buthionine sulfones, penta-, hexa-, heptathionine sulfoximine) in very low tolerable doses (for example pmol to pmol/kg), in addition (metal) chelators (for example α-hydroxy fatty acids, fatty acids (palmitic acids), phytic acid, lactoferrin, EDTA, EGTA), α-hydroxy acids (for example citric acid, lactic acid, malic acid), humic acid, bile acid, bile extracts, bilirubin, biliverdin, unsaturated fatty acids and derivatives thereof (for example palmitoleic acid, α-linolnic acid, γ-linolenic acid, linoleic acid, oleic acid), folic acid and derivatives thereof, ubiquinone and ubiquinol and derivatives thereof, vitamin C and derivatives (for example α-ascorbyl palmitate, Mg—ascorbyl phosphate, ascorbyl acetate), tocopheroles and derivatives (for example vitamin E acetate), vitamin A and derivatives (vitamin A palmitate) and coniferyl benzoate of benzoin gum, rutinic acid and derivatives thereof, ferulic acid and derivatives thereof, butylated hydroxy toluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (for example, ZnO, ZnSO₄), selenium and derivatives thereof (for example selenomethionine), stilbenes and derivatives thereof (for example stilbene oxide, trans-stilbene oxide) and the inventively suitable derivatives (salts, esters, ethers, sugars, nucleotides, nucleosides, peptides and lipids) of said active compounds.

Particular preference is given according to the invention to vitaminn C, vitamin E, vitamin A, plastoquinone, menaquinone, ubiquinols 1-10, ubiquinones 1-10 or derivatives of these substances.

Very particular preference is given to ubiquinols 1-10 and/or derivatives thereof and ubiquinones 1-10 and derivatives thereof Very high preference is given to use of coenzyme Q10.

The amount of the antioxidants (one or more compounds) in the preparations is preferably 0,001 to 30% by weight, particularly preferably 0.05-20% by weight, in particular 1-10% by weight, based on the total weight of the preparation.

If ubiquinone and/or derivatives thereof and/or ubiquinol and/or derivatives thereof, vitamin C and/or derivatives thereof, vitamin E and/or derivatives thereof, vitamin A, or vitamin A derivatives, or carotenes or derivatives thereof are used as antioxidants, it is advantageous to select their respective concentrations from the range 0.001-10% by weight, based on the total weight of the preparation.

The present invention is based on the principle of controlling halitosis by topical application of the abovementioned antioxidants, in particular coenzyme Q10, to the oral and throat mucosa via the usual means. According to the invention, the antioxidants can therefore be used in combination with all customary compositions, in particular the customary oral-care and oral-cleaning preparations known to those skilled in the art. In particular, those which can be used are, in addition to the antioxidants, bactericidal compositions (Triclosan) and/or aroma substances (menthol, menthol derivatives, mint aromas). Surprisingly, the use of said antioxidants leads to a detectable reduction in bad breath odor.

Examples of use are, especially, toothpastes, tooth powders and mouth washes. However, the use of the antioxidants can also come into consideration in any other compositions which are used for treating the oral and throat cavity.

The active compounds according to the invention can be used particularly advantageously in opaque, translucent and transparent microemulsions and/or nanoemulsions. Preparations according to the invention can be used particularly advantageously as

a) unthickened oil-in-water (o/w), bicontinuous or water-in-oil (w/o) micro- and/or nanoemulsions,

b) oil-in-water (o/w), bicontinuous or water-in-oil (w/o) micro- and/or nanoemulsions classically thickened, for example by using polyoxamers, pluronics, carrageenans or vegetable gums,

c) thickened by adding A-B-A triblock copolymers (for example PEG-150-distearate, from Akzo Nobel) or alpha, omega-bis-polyethoxlated silanes or silicones),

d) thickened by adding star polymers (for example PEG-300-pentaerythrityl tetrastearate or hydrophobically modified tetrakis-polyethoxylated silanes and silicones),

e) oil-in-water (o/w), bicontinuous or water-in-oil (w/o) micro- and/or nanoemulsions thickened by adding A-B-A-B multiblock copolymers, starburst polymers, dendrimers and other supramolecular crosslinkers (for example Rheodols, TWIS 399, from KAO, or PEG-120 methylglucose dioleate).

### EXAMPLES

### Formulation Examples

1. Gel Toothpaste Having Activity Against Halitosis

Base: Silica, sodium fluoride

| | I (%) | II (%) | III (%) |
|---|---|---|---|
| Na carboxymethylcellulose | 0.40 | 0.40 | 0.40 |
| Sorbitol, 70% | 72.00 | 72.00 | 72.00 |
| PEG 1500 | 3.00 | 3.00 | 3.00 |
| Na saccharinate | 0.07 | 0.07 | 0.07 |
| Na fluoride | 0.24 | 0.24 | 0.24 |
| PHB ethyl ester | 0.15 | 0.15 | 0.15 |
| Aroma | 1.0 | 1.00 | 1.00 |
| D,L-carnosine | 0.10 | — | — |
| N-acetylcysteine | — | 0.10 | — |
| EDTA | — | — | 0.10 |
| Abrasive silica | 11.00 | 11.00 | 11.00 |
| Silica thickener | 6.00 | 6.00 | 6.00 |
| SDS | 1.40 | 1.40 | 1.40 |
| Distilled water | to 100.00 | to 100.00 | to 100.00 |

2. Antiplacque Toothpaste Having Activity Against Halitosis

Base: Silica, AHP

| | I (%) | II (%) | III (%) |
|---|---|---|---|
| Na carboxymethylcellulose | 1.00 | 1.00 | 1.00 |
| Glycerol 86% | 12.50 | 12.50 | 12.50 |
| Sorbitol 70% | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphosphonic | 1.00 | 1.00 | 1.00 |
| acid, disodium salt (AHP) | | | |
| Bromochlorophen | 0.10 | 0.10 | 0.10 |
| Aroma | 1.10 | 1.10 | 1.10 |
| ZnSO₄ | 0.20 | — | — |
| Coenzyme Q10 | — | 0.20 | — |
| Retinyl palmitate | — | — | 0.20 |
| Abrasive silica | 15.00 | 15.00 | 15.00 |
| Silica thickening | 5.00 | 5.00 | 5.00 |
| SDS | 1.50 | 1.50 | 1.50 |
| Distilled water | to 100.00 | to 100.00 | to 100.00 |

3. Antiplacque Toothpaste Having Activity Against Halitosis

Base: Silica, alkali metal diphosphate

| | I (%) | II (%) | III (%) |
|---|---|---|---|
| Carrageenan | 0.90 | 0.90 | 0.90 |
| Glycerol 86% | 15.00 | 15.00 | 15.00 |
| Sorbitol 70% | 25.00 | 25.00 | 25.00 |
| PEG 1000 | 3.00 | 3.00 | 3.00 |
| Na fluoride | 0.24 | 0.24 | 0.24 |
| Tetrapotassium | 4.50 | 4.50 | 4.50 |
| diphosphate | | | |
| Tetrasodium diphosphate | 1.50 | 1.50 | 1.50 |
| Na saccharinate | 0.40 | 0.40 | 0.40 |
| Silica precipitant | 20.00 | 20.00 | 20.00 |
| Titanium dioxide | 1.00 | 1.00 | 1.00 |
| PHB methyl ester | 0.10 | 0.10 | 0.10 |
| Aroma | 1.10 | 1.10 | 1.10 |
| Lipoic acid | 0.25 | — | — |
| α-Hydroxypalmitic acid | — | 0.25 | — |
| Folic acid | — | — | 0.25 |
| SDS | 1.30 | 1.30 | 1.30 |
| Distilled water | to 100.00 | to 100.00 | to 100.00 |

4. Toothpaste for Sensitive Teeth Having Activity Against Halitosis

Base: Ca carbonate, K nitrate

| | I (%) | II (%) | III (%) |
|---|---|---|---|
| Na carboxymethylcellulose | 0.70 | 0.70 | 0.70 |
| Xanthan gum | 0.50 | 0.50 | 0.50 |
| Glycerol 86% | 15.00 | 15.00 | 15.00 |
| Sorbitol 70% | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Aroma | 1.00 | 1.00 | 1.00 |
| Oleic acid | 0.10 | — | — |
| Glycerol monolaurate | — | 0.10 | — |
| Thiodipropionic acid | — | — | 0.10 |
| Ca carbonate | 35.00 | 35.00 | 35.00 |
| Silicon dioxide | 1.00 | 1.00 | 1.00 |
| SDS | 1.50 | 1.50 | 1.50 |
| Distilled water | to 100.00 | to 100.00 | to 100.00 |

5. Toothpaste for Sensitive Teeth Having Activity Against Halitosis

Base: Silica, strontium chloride

| | I (%) | II (%) | III (%) |
|---|---|---|---|
| Hydroxyethylcellulose | 1.40 | 1.40 | 1.40 |
| Guar gum | 0.60 | 0.60 | 0.60 |
| Glycerol 86% | 18.00 | 18.00 | 18.00 |
| Sorbitol 70% | 12.00 | 12.00 | 12.00 |
| Na saccharinate | 0.35 | 0.35 | 0.35 |
| Dye | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Aroma | 1.20 | 1.20 | 1.20 |
| Urocanic acid | 0.05 | — | — |
| Coenzyme Q10 | — | 0.05 | — |
| Lactic acid | — | — | 0.05 |
| Silica precipitant | 15.00 | 15.00 | 15.00 |
| Silicon dioxide | 1.60 | 1.60 | 1.60 |
| SDS | 1.30 | 1.30 | 1.30 |
| Distilled water | to 100.00 | to 100.00 | to 100.00 |

6. Ready-To-Use Mouthwash Having Fluoride and Activity Against Halitosis

Base: Ethanol, Na fluoride

| | I (%) | II (%) | III (%) |
|---|---|---|---|
| Ethanol 94.7% | 7.00 | 7.00 | 7.00 |
| Glycerol 86% | 12.00 | 12.00 | 12.00 |
| Na fluoride | 0.05 | 0.05 | 0.05 |
| Pluronic F-127 | 1.40 | 1.40 | 1.40 |
| Na phosphate buffer pH | 1.10 | 1.10 | 1.10 |
| 7.0 | | | |
| Sorbic acid | 0.20 | 0.20 | 0.20 |
| Na saccharinate | 0.10 | 0.10 | 0.10 |
| Aroma | 0.15 | 0.15 | 0.15 |
| Ascorbic acid | 0.20 | — | — |
| Trihydroxybutyrophenone | — | 0.20 | — |
| Mannose | — | — | 0.20 |
| Dye | 0.01 | 0.01 | 0.01 |
| Distilled water | to 100.00 | to 100.00 | to 100.00 |

7. Mouthwash Concentrate Having Activity Against Halitosis

Base: Ethanol, aroma

| | I (%) | II (%) | III (%) |
|---|---|---|---|
| Ethanol 94.7% | 80.00 | 80.00 | 80.00 |
| Na cyclamate | 0.15 | 0.15 | 0.15 |
| Aroma | 3.50 | 3.50 | 3.50 |
| Dye | 0.01 | 0.01 | 0.01 |
| Coenzyme Q10 | 0.10 | — | — |
| Distearyl thiodipropionate | — | 0.01 | — |
| Rutic acid | — | — | 0.10 |
| Distilled water | to 100.00 | to 100.00 | to 100.00 |

8. Mouthwash

| | |
|---|---|
| 1,3-di(2-ethylhexyl)cyclohexane | 35.00 |
| Glyceryl lanolate | 5.00 |
| Sorbitan monolaurate | 10.00 |
| Water (+ citric acid to pH 5.5) | 45.00 |

The abovementioned example can also be formulated with an identical amount of retinyl palmitate, tocopheryl acetate. mannose or lycopene, instead of glyceryl lanolate.

9. Mouthwash

| | |
|---|---|
| 1,3-di(2-ethylhexyl)cyclohexane | 33.00 |
| Histidine | 0.50 |
| Sorbitan monolaurate | 10.00 |
| Water (+ citric acid to pH 5.5) | 45.00 |
| PEG-150 distearate | 2.00 |

The abovementioned example can also be formulated using an identical amount of D,L-carnosine, ascorbic acid, humic acid or coenzyme Q10.

10. Throat Wash

| | |
|---|---|
| Steareth-15 | 4.80 |
| Glycerol monostearate | 2.40 |
| Glyceryl lanolate | 2.50 |
| Cyclomethicone | 3.30 |
| Cetearyl octanoate | 1.70 |
| Water | 85.30 |

The abovementioned example can also be formulated using an identical amount of oleic acid monoglyceride, triglycerol monolaurate or glycerol mono palmitate instead of glyceryl lanolate.

11. Throat Wash

| | |
|---|---|
| Steareth-15 | 4.80 |
| Mg ascorbyl phosphate | 0.20 |
| Cholestryl hydroxystearate | 2.50 |
| Cyclomethicone | 3.30 |
| Cetearyl octanoate | 17.00 |
| Water | 83.30 |
| PEG-1500 distearate | 2.00 |

The abovementioned example can also be formulated using an identical amount of coenzyme Q 10, retinyl palmitate, tocopheryl acetate or lycopene, instead of Mg ascorbyl phosphate.

1. The use of antioxidants or antioxidant-containing preparations for preparing compositions for eliminating halitosis or for preventing the formation of halitosis.

2. The use as claimed in claim 1, characterized in that the antioxidants are used in amounts of 0.001-30% by weight (based on the total weight of the preparation).

3. The use as claimed in claim 1, characterized in that the antioxidants are used in amounts of 0.05-20% by weight (based on the total weight of the preparation).

4. The use as claimed in claim 1, characterized in that the antioxidants are used in amounts of 0.05-20% by weight (based on the total weight of the preparation).

5. The use as claimed in claim 1, characterized in that the antioxidants are used in amounts of 1-10% by weight (based on the total weight of the preparation).

6. The use as claimed in claim 1, characterized in that the antioxidants made up are vitamins and coenzymes and derivatives of these substances.

7. The use as claimed in claim 1, characterized in that the antioxidants used are ubiquinone and ubiquinol, vitamin C, vitamin A and vitamin B and derivatives of these substances.

8. The use as claimed in claim 1, characterized in that ubiquinone 1-10, ubiquinol 1-10, menaquinone are used.

9. The use as claimed in one of claim 1, characterized in that a scorbyl palmitate, Mg ascorbyl phosphate, ascorbyl acetate, vitamin E acetate, vitamin A palmitate are used.

10. The use as claimed in one of claim 1, characterized in that coenzyme Q 10 is used.

11. The use as claimed in one of claim 1, characterized in that ubiquinone, ubiquinol, vitamin B, vitamin E, vitamin A, carotenes, carotenoids and the derivatives of these compounds are used in amounts of 0.001-10% by weight (based on the total weight of the preparation).

12. The use as claimed in one of claim 1, characterized in that ubiquinone, ubiquinol, vitamin B, vitamin E, vitamin A, carotenes and derivatives of these compounds are used in amounts of 0.01-5% by weight based on the total weight of the preparation.

13. The use as claimed in one of claim 1, characterized in that ubiquinone, ubiquinol, vitamin B, vitamin E, vitamin A, carotenes and the derivatives of these compounds are used in amounts of 0.1-2.5% by weight based on the total weight of the preparation.
